(19) United States
(12) Patent Application Publication
(10) Pub. No.: US 2022/0323124 A1
(43) Pub. Date: Oct. 13, 2022

(54) FIXATION SYSTEM BETWEEN A MEDICAL DEVICE AND AT LEAST ONE PORTION OF A BONE

(71) Applicants: One Ortho, Saint-Genis-Laval (FR); Universite Savoie Mont Blanc, Chambery (FR); Fianco-Swiss Orthoconsulting, Oberhausbergen (FR)

(72) Inventors: Pierre Vacher, Thorens Glieres (FR); Thibault Goyallon, Annecy (FR); Eric Vittecoq, Groisy (FR); Arnaud Dodelin, Bihorel (FR); Yvan Arlettaz, Monthey (FR); Benoit Schenck, Oberhausbergen (FR); Maxime Antoni, Strasbourg (FR); Christophe Alepee, Lyon (FR); Christophe Depres, Charvonnex (FR)

(21) Appl. No.: 17/616,271

(22) PCT Filed: Apr. 24, 2020
(86) PCT No.: PCT/FR2020/050700

§ 371 Date: Dec. 3, 2021

(30) Foreign Application Priority Data
Jun. 5, 2019 (FR) 1905953

(51) Int. Cl. A61B 17/72

(52) U.S. Cl. CPC A61B 17/7241; A61B 2017/00526

(57) ABSTRACT

The invention relates to a fixation system between a medical device and at least one portion of a bone, characterised in that it comprises an elongate portion intended to be inserted into the bone, in particular into the medullary cavity and/or into the proximal or distal ends, the elongate portion having a wall consisting of meshes through which at least one fixing member can pass, according to a position and orientation that are not predefined, and which apply, by elastic, plastic or elastic-plastic deformation, a transverse and/or axial pressure force onto the fixing member, the fixing member being intended to also pass through the portion of the bone in order to ensure rigid fixation between the bone and the medical device.

### TECHNICAL FIELD

The present invention relates to the technical field of orthopaedic surgery, and more specifically to a fixation system between a medical device and at least one portion of a bone.

By medical device, this means any device intended to remain in the body of a patient in the long term, such as a trauma implant, or an orthopaedic implant with a prosthetic stem, or an intramedullary nail, for example.

### PRIOR ART

It is well-known for a person skilled in the art to use different devices, commonly called fitting ancillaries, for locking and fixing an implant to a bone, or vice versa for fixing bone fragments to an implant.

For example, for intramedullary nailing, the intramedullary nail has a plurality of guide orifices, with predetermined angular positions and heights, which are intended to be passed through by fixing screws which are screwed into the corresponding part of the bone in order to lock the intramedullary nail in position. To do this, the surgeon has a fitting ancillary, equipped with an external viewfinder making it possible to align a drilling device in order to produce complementary orifices in the portion of the bone in order to be able to engage fixing screws through the bone and the intramedullary nail.

The disadvantages of this technique reside in the need to use, on the one hand, a fitting ancillary in order to know the drilling axes and the axes of the orifices intended to receive the fixing screws and, on the other hand, an inspection radiograph, in order to verify the correct alignment and the correct engagement of the fixing screws.

This technique is relatively complex, and presents risks for the surgeon of not controlling the alignment between the drillings of the bone and the orifices of the intramedullary nail, such that the fixing screws cannot be engaged.

It is also known to a person skilled in the art, in order to return bone fragments to position and in compression on a bone, to use sutures intended to be knotted around the bone and bone fragments.

This technique has the disadvantage that the sutures tend to perform a vascular laceration, which causes necrosis due to lack of irrigation of the bone.

It is known from the prior art, that document D1 shows meshes after a structure has been deformed in a bone volume, and therefore there is no control over the size of the meshes, and in addition, once the structure has been deformed, it can no longer be extracted from a patient.

### DISCLOSURE OF THE INVENTION

One of the aims of the invention is thus to overcome the abovementioned disadvantages by proposing a fixation system between a medical device and at least one portion of a bone, possibly making it possible to avoid using a fitting ancillary or of an external viewfinder, while making it possible to reduce or even avoid the use of sutures and therefore to reduce or even eliminate the risk of necrosis.

To this end, and according to the invention, a system has been developed for fixing a medical device to at least one portion of a bone, noteworthy in that the medical device comprises an elongate portion, for example made of a biocompatible polymer or metal, intended to be inserted into the bone, in particular into the medullary cavity and/or into the proximal or distal ends. The elongate portion has a wall constituted by meshes, preferably non-expandable, adapted to be passed through by at least one fixing member, according to a position and an orientation that are not predefined, while exerting, by elastic, plastic or elastic-plastic deformation, a pressure force, for example axial and/or transverse, on said fixing member. The fixing member is also intended to pass through the portion of the bone in order to ensure a rigid fixation between the bone and the medical device.

In this way, in order to fix a portion of a bone and the medical device together, the invention proposes to use a fixing member, such as a screw, a tip, or a clip, optionally in combination with a wire, and to cause this fixing member to pass through the portion of the bone to be fixed and the medical device. The elongate portion of the medical device, preferably rectilinear, is designed in particular to be passed through by at least one fixing member in a position and an orientation that are not predefined.

Thus, the advantage of the present invention resides in the fact that the fixing member passes through the meshes of the elongate portion of the medical device in a position that is not predefined, whether in a diametral plane or eccentric with respect to the longitudinal axis of the elongate portion, in an orthogonal direction or with an incidence. In this way, the surgeon can freely choose the number and the position of the fixing members to be used, and can freely choose the technique for fixing the bone portion, for example by clips, by screws, by tips, etc. By position and orientation that are not predefined, it is understood that the surgeon can themselves decide, during the intervention, the orientation and the position of the fixing member, and he have a plurality of choices for the orientation and the positioning of the fixing member.

Another advantage therefore resides in the fact that the use of a fitting ancillary or of an inspection radiograph is no longer necessary, although in practice, the surgeon can still use it to find the orientation of a fixing member already inserted.

The meshes are non-expandable, i.e. they are fixed and rigid such that they are passed through by the fixing member while exerting, by deformation, a pressure force on said fixing member. This pressure force is, for example, transverse and generated by transverse deformation of the meshes of the elongate portion of the medical device. In this manner, the transverse pressure force exerted on the fixing member allows it to be held in position and locked and, consequently, the portion of the bone to be fixed to said medical device, or the medical device to be fixed to the bone.

For example, in the case of a fixing member in the form of a screw, the screw passes through the portion of the bone, and is screwed inside the elongate portion of the medical device in order to fix them together. For optimal fixation, the screw passes through the elongate portion of the medical device and is anchored in the bone.

In the case of a fixing member in the form of a screw, the meshes preferably have thicknesses substantially greater than the pitch of the screw, for example a few hundredths or tenths of a millimetre, in order to exert, by axial deformation of the thickness of the meshes, an axial pressure force on the screw when it passes through the meshes.

The meshes are, for example, defined by a lattice formed by a plurality of rigid strands. The lattice is made, for example, of a bio-compatible polymer or metal, preferably with strands of circular cross-sections.

The elongate portion is, for example, tubular, and is advantageously in the form of a part produced by a three-dimensional printing technique. Thus, it is easy to produce the lattice with rigid strands with circular cross-sections. Furthermore, the ends of the strands are fixedly connected to each other. The three-dimensional printing technique makes it possible to easily produce this embodiment and to completely control the shape dimension and the dimension of the meshes in order to completely adjust the pressure forces, transverse and/or axial, on the fixing member .

In order to avoid bone growth inside the elongate portion, the elongate portion is coated with a film or filled with a material which does not oppose the passage of the fixing members, but which prevents bone growth.

According to different embodiments, the medical device consists entirely of the elongate portion itself, intended to be inserted into the medullary cavity of the bone and/or to be housed in the proximal or distal end of the bone, in order to fix bone fragments therein after immobilisation on a portion of the healthy bone.

The medical device can also be an intramedullary nail, or a prosthetic stem, comprising, on their part intended to be inserted into the medullary cavity of the bone, the elongate portion according to the invention, in order to be passed through by fixing members and to lock said intramedullary nail or the prosthetic stem in position with respect to the part of the healthy bone in which they are implanted.

The medical device can also be an intramedullary nail, or a prosthetic stem, comprising, on their part intended to be housed in the proximal or distal ends of the bone, the elongate portion according to the invention, in order to be passed through by fixing members and thus fix the bone fragments on said intramedullary nail or the prosthetic stem.

### BRIEF DESCRIPTION OF THE DRAWINGS

Other characteristics and advantages of the invention will become apparent from the following description, which is given by way of non-limiting indication and made with reference to the appended figures, in which:

FIG. 1 shows, in a longitudinal cross-section, a bone with the elongate portion of a medical device inserted in the medullary canal, and screws enabling a part of the bone to be fixed to the medical device by locking inside the elongate portion;

FIG. 2 is a perspective schematic view, showing the elongate portion of the medical device with a wall formed by a lattice formed from a plurality of braided wires;

FIG. 3 is a view similar to that of FIG. 2, also showing fixing screws passing throughout the lattice of the elongate portion.

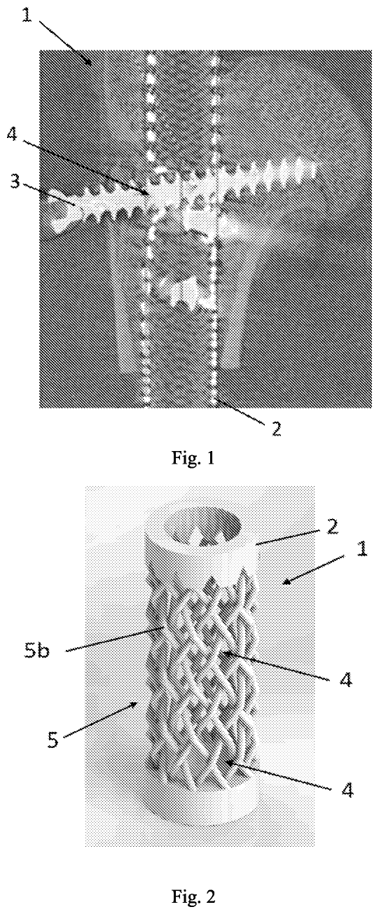

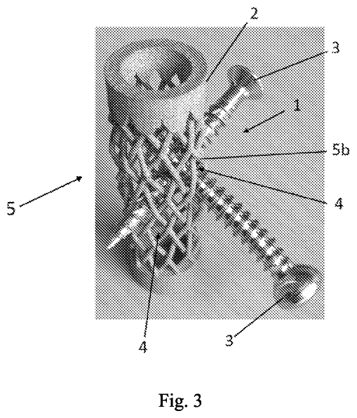

### DETAILED DESCRIPTION OF THE INVENTION

The invention relates to a system for fixing a medical device (1) to at least one portion of a bone. The invention has, for example, an advantageous application for resetting and compressing bone fragments on a bone, but also for fixing to a bone, any type of medical device (1) intended to be inserted into the medullary cavity of a bone and/or to be housed in the proximal or distal end of the bone.

To this end, according to the invention, the medical device (1) intended to be fixed to a bone, or to which bone fragments are intended to be fixed, comprises an elongate portion (2) intended to be inserted into the medullary cavity of the bone and/or to be housed in the proximal or distal ends.

The elongate portion (2) can either constitute the medical device (1) itself, when it is intended to fix bone fragments thereto, or can constitute a part of a medical device (1), for example, a part of a prosthetic stem or of an intramedullary nail, which are intended to be inserted into the medullary cavity and fixed to the bone.

To this end, the elongate portion (2), which is preferably tubular, has a wall consisting of non-expandable meshes (4) adapted to be passed right through, by one or more fixing members (3), such as screws, clips, tips, etc., according to a position and an orientation that are not predefined, and by exerting, by deformation, a pressure force, in particular transverse and/or axial, on said fixing members (3).

The meshes (4) are, for example, defined by a lattice (5) formed by a plurality of rigid strands (5b). The rigid strands (5b) can be braided and independent of each other at the intersections between the different strands (5b).

In this way, it is possible to produce a rigid, non-expandable elongate portion (2) with meshes (4) of controlled dimensions, while making it possible to adapt to any type of fixing member (3), whatever its orientation, since, given that the strands (5b) are independent of one another at the intersections, the strands can move apart while continuing to exert the pressure force against the fixing member .

In another configuration, the rigid strands (5b) can be braided and secured to one another at the intersections between the different strands (5b).

The invention thus makes it possible to fix the medical device (1) to a portion of a bone, or bone fragments on the medical device (1), by means of the fixing members (3) passing through the bone and the elongate portion (2) of the medical device (1).

The invention makes it possible for the surgeon to freely choose the position (height, inclination and eccentricity) of the fixing members (3), while being able to avoid using a fitting ancillary or an inspection radiograph. The fixing members (3) can pass through the meshes (4) of the elongate portion (2) in a position and an orientation which are not predefined, i.e. not necessarily in a diametral plane, not necessarily perpendicular to the axis of the elongate portion, at the discretion of the surgeon.

In practice, during use, a fixing member (3) passes through a portion of the bone and then passes right through the elongate portion (2), penetrating successively into two meshes (4), which exert a pressure, in particular transverse and/or axial, on said fixing member (3), in particular because one of their dimensions is in a tight fit with the fixing member (3). For example, when the width of the meshes (4) is in a tight fit, that is to say substantially less by a few tenths or hundredths of a millimetre with respect to the section of the fixing member (3), the meshes (4) exert a transverse pressure on the fixing member (3). For example, when the fixing member (4) is in the form of a screw, one of the dimensions of the meshes (4), in particular the width of the meshes (4), can be in a tight fit with the thread root diameter of the screw in order to exert a transverse pressure on the screw.

Alternatively or in combination, the thickness of the meshes (4) can be in a tight fit, i.e. substantially greater by a few hundredths or tenths of a millimetre with respect to the screw thread so as to exert, by deformation, an axial force on the screw.

According to one particular embodiment, the elongate portion (2) is produced, for example, from a planar grid wound to have a generally cylindrical shape, with or without axial welding.

An advantageous technique consists, in reference to the figures, in manufacturing, by additive manufacture, layer by layer, a tube, a portion of which is produced by a lattice (5) with a plurality of rigid strands (5b), for example with circular cross-sections.

Moreover, the ends of the strands (5b) are fixedly connected to one another at the two ends of the elongate portion (2). The three-dimensional printing technique makes it possible to easily produce this embodiment and to completely control the shape and the dimension of the meshes (4) in order to completely adjust the transverse and/or axial pressure forces on the fixing member.

The geometry of the meshes (4) is adapted to that of the fixing members (3). This geometry makes it possible to generate, by the deformation of the meshes (4), a transverse and/or axial pressure on the fixing members (3), making it possible to lock them in the elongate portion (2). The insertion forces of said fixing members (3) must remain moderate, while ensuring an adjusted assembly, without clearance, allowing a slight deformation of the mesh (4).

In order to facilitate disassembly, the elongate portion (2) can be coated with a film, not shown, to prevent bone growth within said elongate portion (2), or can be filled with a material which does not oppose the passage of the fixing members (3), but which prevents bone growth.

According to different embodiments, the meshes (4) can be made by laser multi-drilling the elongate portion (2), or else from an expanded metal, without moving away from the scope of the invention.

It is clear from the above that the invention does indeed provide a system for fixing a medical device (1) to at least one portion of a bone, possibly making it possible to avoid using an fitting ancillary or of an external viewfinder. To carry out the fixing, the surgeon chooses the number and the orientation of the fixing members (3), being certain to be able to penetrate the elongate portion (2) to carry out the locking. The invention makes it possible to fix bone fragments, while making it possible to reduce or even avoid the use of sutures and thus to reduce or even eliminate the risk of necrosis.

1. A medical device comprising a system for fixing to at least one portion of a bone, wherein the medical device comprises an elongate portion intended to be inserted in the bone, in particular in the medullary cavity and/or in the proximal or distal ends, the elongate portion has a wall consisting of meshes designed to be passed through by at least one fixing member, in a position and an orientation that are not predefined, while exerting by elastic, plastic or elastoplastic deformation, a transverse and/or axial pressure force on said fixing member, the fixing member being intended to also pass through the portion of the bone to ensure rigid fixing between the bone and the medical device.

2. The medical device according to claim 1, wherein the meshes are defined by a lattice formed by a plurality of rigid strands.

3. The medical device according to claim 2, wherein the lattice is made with strands with circular cross-sections.

4. The medical device according to claim 1 wherein the elongate portion is tubular.

5. The medical device according to claim 1, wherein the elongate portion is coated with a film to prevent bone growth inside said elongate portion or filled with a material which does not oppose the passage of the fixing members, but which prevents bone growth.

6. The medical device according to claim 1, wherein the fixing member is a screw.

7. The medical device according to claim 1, wherein one of the dimensions of the meshes is in a tight fit with the cross-section of the fixing member so as to exert a transverse pressure on the fixing member.

8. The medical device according to claim 6, wherein the meshes have thicknesses in tight fit with the pitch of the screw so as to exert an axial pressure force on the screw as it passes through the meshes.

9. The medical device according to claim 1 wherein the medical device is an intramedullary nail or a prosthetic stem.

10. A method for manufacturing the medical device of claim 1, wherein the method consists of producing the elongate portion by a three-dimensional printing technique.
